(19) United States
(12) Patent Application Publication
(10) Pub. No.: US 2024/0335630 A1
(43) Pub. Date: Oct. 10, 2024

(54) SYSTEMS AND METHODS FOR CHARACTERIZING A CONDUIT IN A RESPIRATORY THERAPY SYSTEM

(71) Applicant: ResMed Digital Health Inc., San Diego, CA (US)

(72) Inventors: Stephen MCMAHON, Dublin (IE); Redmond SHOULDICE, Dublin (IE)

(21) Appl. No.: 18/574,684

(22) PCT Filed: Jun. 30, 2022
(86) PCT No.: PCT/IB2022/056098

§ 371 Date: Dec. 27, 2023

Related U.S. Application Data
(60) Provisional application No. 63/217,228, filed on Jun. 30, 2021.

(51) Int. Cl. A61M 16/00; A61M 16/08; A61M 16/16; A61M 16/20

(52) U.S. Cl. CPC A61M 16/024; A61M 16/0875; A61M 16/16; A61M 16/208; A61M 2016/0027; A61M 2016/0033; A61M 2205/14; A61M 2205/15

(57) ABSTRACT

Various implementations of the present disclosure are directed to systems and methods for characterizing a conduit coupled to a respiratory therapy device in a respiratory therapy system. The method includes generating a first airflow from the respiratory therapy device to the conduit by operating a motor of the respiratory therapy device at a first revolutions per minute. The method further includes receiving a first airflow parameter data associated with the first airflow, wherein the first airflow parameter data has a first pair of two distinct airflow parameter values. The method further includes determining a first relationship between the first airflow parameter data and the conduit, and characterizing the conduit based on the first relationship.

### PRIORITY CLAIM

The present disclosure claims benefit of and priority to U.S. Provisional Ser. No. 63/217,228 filed Jun. 30, 2021. The contents of that application are hereby incorporated by reference in their entirety.

### TECHNICAL FIELD

The present disclosure relates generally to respiratory therapy systems, and more particularly, to systems and methods for characterizing a conduit coupled to a respiratory therapy device in a respiratory therapy system by determining a relationship between the conduit and parameter data of airflow through the conduit.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), hypopneas and other types of apneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system.

Current flow management algorithms continue to seek better control of airflow from a respiratory therapy device coupled to a user interface through a conduit of the respiratory therapy system. Such control of airflow can be guided by accurate characterization of the conduit and optionally the user interface coupled thereto, when the respiratory therapy device is not being used by a user for therapy. Accordingly, it is desirable to have systems and methods for characterizing the conduit such that pressurized airflow delivered to the user during therapy can be controlled and optimized.

### SUMMARY

According to some implementations of the present disclosure, an example method for characterizing a conduit coupled to a respiratory therapy device in a respiratory therapy system is disclosed. The method includes generating a first airflow from the respiratory therapy device to the conduit by operating a motor of the respiratory therapy device at a first revolutions per minute. The method further includes receiving a first airflow parameter data associated with the first airflow, wherein the first airflow parameter data has a first pair of two distinct airflow parameter values. The method further includes determining a first relationship between the first airflow parameter data and the conduit, and characterizing the conduit based on the first relationship.

A further implementation of the example method is where the characterizing the conduit comprises determining (i) presence or absence of the conduit, (ii) a type of the conduit, (iii) presence or absence of a user interface coupled to the conduit, (iv) determining a type of the user interface coupled to the conduit, or (v) any combination thereof. Another implementation is where the type of conduit is characterized as having a specific inner diameter. Another implementation is where the specific inner diameter is in a range between 8-20 mm. Another implementation is where the type of conduit is one of: (i) a conduit having an inner diameter of about 12 mm, (ii) a conduit having an inner diameter of about 15 mm, or (iii) a conduit having an inner diameter of about 19 mm. Another implementation is where the type of user interface is one of (i) a full face mask, (ii) a nasal mask, or (iii) a nasal pillows mask. Another implementation is where the first airflow parameter data comprises a plurality of first pairs of airflow parameter values associated with the first airflow. Another implementation is where the first pair of airflow parameter values includes any two of: (i) a pressure value, (ii) a flow rate value, and (iii) an impedance value calculated as a ratio of the pressure value and the flow rate value of the first airflow. Another implementation is where the first pair of airflow parameter values includes (i) a laminar flow constant and (ii) a turbulent flow constant. Another implementation is where the first relationship is a linear relationship between the laminar flow constant and the turbulent flow constant for a given pressure value and a given flow rate of the first airflow. Another implementation is where the determining the first relationship further includes estimating a first measure of central tendency associated with the first airflow parameter data. Another implementation is where the example method further includes characterizing the conduit based on the first measure of central tendency associated with the first airflow parameter data satisfying a first condition. Another implementation is where the first measure of central tendency is (i) a mean of the first airflow parameter data, (ii) a median of the first airflow parameter data, or (iii) a mode of the first airflow parameter data. Another implementation is where the estimating the first measure of central tendency excludes an airflow parameter value that is at least two standard deviations away from the mean of the first airflow parameter data. Another implementation is where the satisfying the first condition includes exceeding a first threshold value, not exceeding the first threshold value, staying within a first predetermined range of values, staying outside the first predetermined range of values, or any combination thereof. Another implementation is where both the first threshold value and the first predetermined range of values are associated with (i) a pressure value, (ii) a flow rate value, and (iii) an impedance value calculated as a ratio of the pressure value and the flow rate value at the first revolutions per minute. Another implementation is where the first airflow is generated for not more than 5 seconds. Another implementation is where the example method further includes generating a second airflow from the respiratory therapy device to the conduit by operating the motor of the respiratory therapy device at a second revolutions per minute. The example method also includes receiving a second airflow parameter data associated with the second airflow, the second airflow parameter data comprising a second pair of two distinct airflow parameter values. The example method further includes determining a second relationship between the second airflow parameter data and the conduit. Another implementation is where at least one of (i) the first airflow and (ii) the second airflow has a Reynolds number between about 2300 and about 4000. Another implementation is where the second airflow parameter data comprises a plurality of second pairs of airflow parameter values associated with the second airflow. Another implementation is where the second pair of airflow parameter values includes any two of: (i) a pressure value, (ii) a flow rate value, and (iii) an impedance value calculated as a ratio of the pressure value and the flow rate value of the second airflow. Another implementation is where the second pair of airflow parameter values includes (i) a laminar flow constant and (ii) a turbulent flow constant. Another implementation is where the second relationship is a linear relationship between the laminar flow constant and the turbulent flow constant for a given pressure value and a given flow rate of the second airflow. Another implementation is where the characterizing the conduit is further based on the first pair of airflow parameter values and the second pair of airflow parameter values. Another implementation is where the determining the second relationship further includes estimating a second measure of central tendency associated with the second airflow parameter data. The characterizing the conduit is further based on the second measure of central tendency associated with the second airflow parameter data satisfying a second condition. Another implementation is where the second measure of central tendency is (i) a mean of the second airflow parameter data, (ii) a median of the second airflow parameter data, or (iii) a mode of the second airflow parameter data. Another implementation is where the estimating the second measure of central tendency excludes an airflow parameter value that is at least two standard deviations away from the mean of the second airflow parameter data. Another implementation is where the satisfying the second condition includes exceeding a second threshold value, not exceeding the second threshold value, staying within a second predetermined range of values, staying outside the second predetermined range of values, or any combination thereof. Another implementation is where the second threshold value and the second predetermined range of values are associated with (i) a pressure value, (ii) a flow rate value, and (iii) an impedance value calculated as a ratio of the pressure value and the flow rate value at the second revolutions per minute. Another implementation is where the example method further includes estimating a first slope of a line through the first pair of airflow parameter values and the second pair of airflow parameter values. Characterizing the conduit is further based on the first slope satisfying a third condition. Another implementation is where the example method further includes estimating a second slope of a line through the first and the second measures of central tendency associated with the first airflow parameter data and the second airflow parameter data respectively. Characterizing the conduit is further based on the second slope satisfying the third condition. Another implementation is where the satisfying the third condition includes exceeding a third threshold value, not exceeding the third threshold value, staying within a third predetermined range of values, staying outside the third predetermined range of values, or any combination thereof. Another implementation is where the first airflow and the second airflow are generated for not more than 5 seconds. Another implementation is where the characterizing the conduit is implemented using a classification-based machine learning algorithm using training data labeled with characterizations of the conduit. Another implementation is where the classification-based machine learning algorithm uses a KNN (K-Nearest Neighbor) technique. Another implementation is where the characterizing the conduit is associated with a confidence interval that the characterization of the conduit is correct. Another implementation is where the characterizing the conduit is compared against a characterization of the conduit obtained through an acoustic signature of airflow through the conduit. Another implementation is where the flow rate value of the first airflow and the second airflow is generated by a flow sensor communicatively coupled to the respiratory therapy device. Another implementation is where the pressure value of the first airflow and the second airflow is generated by a pressure sensor communicatively coupled to the respiratory therapy device. Another implementation is where at least one or both of (i) the first airflow, and (ii) the second airflow is a laminar flow of air. Another implementation is where the laminar flow of air has a Reynolds number of less than about 2300. Another implementation is where at least one or both of (i) the first airflow, and (ii) the second airflow has an associated noise level of no more than about 40 dBA measured at a distance of about one meter from the respiratory therapy device. Another implementation is where at least one or both of (i) the first airflow, and (ii) the second airflow has a flow rate value of between about 0.1 and 0.8 liters per second. Another implementation is where the motor of the respiratory therapy device operates between about 3.000 revolutions per minute and about 12.000 revolutions per minute. Another implementation is where the example method includes determining, based on characterizing the conduit, a third airflow parameter value for prospective delivery of pressurized airflow to a user, when the user wears the user interface for respiratory therapy. Another implementation is where the determining the third airflow parameter value further comprises determining impedance due to (i) intentional leak through one or more vents in the user interface, (ii) unintentional leak at the user interface or a mouth of the user, or (iii) a combination of both. Another implementation is where the user interface includes an anti-asphyxia valve. At least one of (i) the first airflow, and (ii) the second airflow has a flow rate value of less than about 0.4 liters per second. Another implementation is where the respiratory therapy device includes a waterless humidifier. The example method further includes determining a type of the waterless humidifier based on the first relationship.

Another disclosed example is a system including a control system comprising one or more processors; and a memory having stored thereon machine-readable instructions. The control system is coupled to the memory. Any of the above described methods are implemented when the machine-readable instructions in the memory are executed by at least one of the one more or processors of the control system.

Another disclosed example is a system for communicating one or more indications to a user. The system includes a control system configured to implement any of the above described methods.

Another disclosed example is a computer program product comprising instructions which, when executed by a computer, cause the computer to carry out any of the methods of any one of the above described claims. Another implementation is where the computer program product is a non-transitory computer readable medium.

According to some implementations of the present disclosure, an example system for characterizing a conduit coupled to a respiratory therapy device in a respiratory therapy system is disclosed. The system includes a respiratory therapy device configured to supply pressurized air during a sleep session of a user and one or more sensors configured to detect airflow parameter data associated with the supplied pressurized air. The system further includes a control system including one or more processors configured to execute machine-readable instructions to generate a first airflow from the respiratory therapy device to the conduit by operating a motor of the respiratory therapy device at a first revolutions per minute. The control system is further configured to receive a first airflow parameter data associated with the first airflow: wherein the first airflow parameter data has a first pair of two distinct airflow parameter values. The control system is further configured to determine a first relationship between the first airflow parameter data and the conduit, and characterize the conduit based on the first relationship.

A further implementation of the example system is where characterizing the conduit comprises determining (i) presence or absence of the conduit, (ii) a type of the conduit, (iii) presence or absence of a user interface coupled to the conduit, (iv) determining a type of the user interface coupled to the conduit, or (v) any combination thereof. Another implementation is where the type of conduit is characterized as having a specific inner diameter. Another implementation is where the specific inner diameter is in a range between 8-20 mm. Another implementation is where the type of conduit is one of: (i) a conduit having an inner diameter of about 12 mm, (ii) a conduit having an inner diameter of about 15 mm, or (iii) a conduit having an inner diameter of about 19 mm. Another implementation is where the type of user interface is one of (i) a full face mask, (ii) a nasal mask, or (iii) a nasal pillows mask. Another implementation is where the first airflow parameter data comprises a plurality of first pairs of airflow parameter values associated with the first airflow. Another implementation is where the first pair of airflow parameter values includes any two of: (i) a pressure value, (ii) a flow rate value, and (iii) an impedance value calculated as a ratio of the pressure value and the flow rate value of the first airflow. Another implementation is where the first pair of airflow parameter values includes (i) a laminar flow constant and (ii) a turbulent flow constant. Another implementation is where the first relationship is a linear relationship between the laminar flow constant and the turbulent flow constant for a given pressure value and a given flow rate of the first airflow. Another implementation is where the determining the first relationship further includes estimating a first measure of central tendency associated with the first airflow parameter data. Another implementation is where the example control system characterizes the conduit based on the first measure of central tendency associated with the first airflow parameter data satisfying a first condition. Another implementation is where the first measure of central tendency is (i) a mean of the first airflow parameter data, (ii) a median of the first airflow parameter data, or (iii) a mode of the first airflow parameter data. Another implementation is where the estimating the first measure of central tendency excludes an airflow parameter value that is at least two standard deviations away from the mean of the first airflow parameter data. Another implementation is where the satisfying the first condition includes exceeding a first threshold value, not exceeding the first threshold value, staying within a first predetermined range of values, staying outside the first predetermined range of values, or any combination thereof. Another implementation is where both the first threshold value and the first predetermined range of values are associated with (i) a pressure value, (ii) a flow rate value, and (iii) an impedance value calculated as a ratio of the pressure value and the flow rate value at the first revolutions per minute. Another implementation is where the first airflow is generated for not more than 5 seconds. Another implementation is where the example control system is further configured to generate a second airflow from the respiratory therapy device to the conduit by operating the motor of the respiratory therapy device at a second revolutions per minute. The example control system is further configured to receive a second airflow parameter data associated with the second airflow, the second airflow parameter data comprising a second pair of two distinct airflow parameter values. The example control system is further configured to determine a second relationship between the second airflow parameter data and the conduit. Another implementation is where at least one of (i) the first airflow and (ii) the second airflow has a Reynolds number between about 2300 and about 4000. Another implementation is where the second airflow parameter data comprises a plurality of second pairs of airflow parameter values associated with the second airflow. Another implementation is where the second pair of airflow parameter values includes any two of: (i) a pressure value, (ii) a flow rate value, and (iii) an impedance value calculated as a ratio of the pressure value and the flow rate value of the second airflow. Another implementation is where the second pair of airflow parameter values includes (i) a laminar flow constant and (ii) a turbulent flow constant. Another implementation is where the second relationship is a linear relationship between the laminar flow constant and the turbulent flow constant for a given pressure value and a given flow rate of the second airflow. Another implementation is where the characterizing the conduit is further based on the first pair of airflow parameter values and the second pair of airflow parameter values. Another implementation is where the determining the second relationship further includes estimating a second measure of central tendency associated with the second airflow parameter data. The characterizing the conduit is further based on the second measure of central tendency associated with the second airflow parameter data satisfying a second condition. Another implementation is where the second measure of central tendency is (i) a mean of the second airflow parameter data, (ii) a median of the second airflow parameter data, or (iii) a mode of the second airflow parameter data. Another implementation is where the estimating the second measure of central tendency excludes an airflow parameter value that is at least two standard deviations away from the mean of the second airflow parameter data. Another implementation is where the satisfying the second condition includes exceeding a second threshold value, not exceeding the second threshold value, staying within a second predetermined range of values, staying outside the second predetermined range of values, or any combination thereof. Another implementation is where the second threshold value and the second predetermined range of values are associated with (i) a pressure value, (ii) a flow rate value, and (iii) an impedance value calculated as a ratio of the pressure value and the flow rate value at the second revolutions per minute. Another implementation is where the example control system is configured to estimate a first slope of a line through the first pair of airflow parameter values and the second pair of airflow parameter values. Characterizing the conduit is further based on the first slope satisfying a third condition. Another implementation is where the example control system is configured to estimate a second slope of a line through the first and the second measures of central tendency associated with the first airflow parameter data and the second airflow parameter data respectively. Characterizing the conduit is further based on the second slope satisfying the third condition. Another implementation is where the satisfying the third condition includes exceeding a third threshold value, not exceeding the third threshold value, staying within a third predetermined range of values, staying outside the third predetermined range of values, or any combination thereof. Another implementation is where the first airflow and the second airflow are generated for not more than 5 seconds. Another implementation is where the characterizing the conduit is implemented using a classification-based machine learning algorithm using training data labeled with characterizations of the conduit. Another implementation is where the classification-based machine learning algorithm uses a KNN (K-Nearest Neighbor) technique. Another implementation is where the characterizing the conduit is associated with a confidence interval that the characterization of the conduit is correct. Another implementation is where the characterizing the conduit is compared against a characterization of the conduit obtained through an acoustic signature of airflow through the conduit. Another implementation is where the flow rate value of the first airflow and the second airflow is generated by a flow sensor communicatively coupled to the respiratory therapy device. Another implementation is where the pressure value of the first airflow and the second airflow is generated by a pressure sensor communicatively coupled to the respiratory therapy device. Another implementation is where at least one or both of (i) the first airflow, and (ii) the second airflow is a laminar flow of air. Another implementation is where the laminar flow of air has a Reynolds number of less than about 2300. Another implementation is where at least one or both of (i) the first airflow, and (ii) the second airflow has an associated noise level of no more than about 40 dBA measured at a distance of about one meter from the respiratory therapy device. Another implementation is where at least one or both of (i) the first airflow, and (ii) the second airflow has a flow rate value of between about 0.1 and 0.8 liters per second. Another implementation is where the motor of the respiratory therapy device operates between about 3,000 revolutions per minute and about 12,000 revolutions per minute. Another implementation is where the example control system is configured to determine, based on characterizing the conduit, a third airflow parameter value for prospective delivery of pressurized airflow to a user, when the user wears the user interface for respiratory therapy. Another implementation is where the determining the third airflow parameter value further comprises determining impedance due to (i) intentional leak through one or more vents in the user interface, (ii) unintentional leak at the user interface or a mouth of the user, or (iii) a combination of both. Another implementation is where the user interface includes an anti-asphyxia valve. At least one of (i) the first airflow; and (ii) the second airflow has a flow rate value of less than about 0.4 liters per second. Another implementation is where the example system includes a waterless humidifier coupled to the respiratory therapy device. The control system is further configured to determine a type of the waterless humidifier based on the first relationship.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a respiratory therapy system, according to some implementations of the present disclosure.

FIG. 2 is a perspective view of at least a portion of the system of FIG. 1, a user, and a bed partner, according to some implementations of the present disclosure.

FIGS. 3A-3C illustrate perspective and cross-sectional views of different types of conduits having different configurations, according to some implementations of the present disclosure.

FIGS. 4A-4C illustrate perspective views of different types of user interfaces configured to be coupled to a conduit in the respiratory therapy system, according to some implementations of the present disclosure.

FIG. 5A illustrates a graphical representation of values of impedance and pressure plotted over values of flow rate through a conduit in the respiratory therapy system, according to some implementations of the present disclosure.

FIG. 5B illustrates a graphical representation of Reynolds number indicating laminar flow, transition flow, and turbulent flow plotted over values of flow rate corresponding to FIG. 5A, according to some implementations of the present disclosure.

FIG. 6A shows a circuit diagram of an open airflow circuit between the respiratory therapy device and the conduit in the respiratory therapy system, according to some implementations of the present disclosure.

FIG. 6B shows a graphical representation of values of pressure drop over values of flow rate through different types of user interface coupled to a conduit having an inner diameter of about 15 mm and placed in the open flow circuit of FIG. 6A, according to some implementations of the present disclosure.

FIG. 7 illustrates a graphical representation of the values of impedance over values of flow rate through different types of conduits that are not coupled to a user interface in the respiratory therapy system, according to some implementations of the present disclosure.

FIG. 8 illustrates a graphical representation of the values of impedance over values of flow rate through different types of user interfaces coupled to a conduit having an inner diameter of about 15 mm in the respiratory therapy system, according to some implementations of the present disclosure.

FIG. 9 illustrates a graphical representation of the values of impedance over values of flow rate through different types of user interfaces, with or without an anti-asphyxia valve, coupled to a conduit having an inner diameter of about 15 mm in the respiratory therapy system, according to some implementations of the present disclosure.

FIG. 10 illustrates improved resolution of airflow parameter data in FIG. 9 due to data collected in the laminar flow, transition flow, and turbulent flow regions, according to some implementations of the present disclosure.

FIG. 11 illustrates a graphical representation of the values of laminar flow constant over values of turbulent flow constant measured for different configurations of conduits with or without being coupled to a user interface in the respiratory therapy system, according to some implementations of the present disclosure.

FIG. 12 illustrates a flow diagram for a method of characterizing a conduit coupled to a respiratory therapy device in the respiratory therapy system, according to some implementations of the present disclosure.

FIG. 13 illustrates a graphical representation of the values of impedance over values of flow rate through different types of conduits that may indicate the presence and type of a waterless humidifier in a respiratory therapy system, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Various embodiments of the present disclosure are directed to systems and methods for characterizing a conduit coupled to a respiratory therapy device, and optionally a user interface coupled to the conduit in a respiratory therapy system, by determining a relationship between the conduit and parameter data of airflow through the conduit. Characterization of the conduit can include determining presence or absence of the conduit, determining a type of the conduit, determining presence or absence of a user interface coupled to the conduit, determining a type and specific manufacturer and model of the user interface, and whether the user interface includes an anti-asphyxia valve (AAV). The methods and systems can be implemented when the respiratory therapy device is not being used by a user for therapy and the user interface is not yet engaged with the face of a user (e.g., before or after therapy, before a first use). Advantageously, the methods and systems described herein can be implemented quickly and quietly, without complexity of computation and without being confounded by a leak or breathing of the user.

The methods and systems allow this characterization at relatively low flow rates of airflow; which is quiet and effectively imperceptible to a user. At low flow rates between about 0.1-0.4 liters per second (L/s), airflow parameter values show unique and distinguishable signatures that help accurately characterize the conduit and the user interface coupled thereto. For example, the laminar flow constant K_{L} and the turbulent flow constant, K_{T} are more non-linear at the low flow rates, and can be used to distinguish and characterize different conduit and conduit-user interface combinations. As another example, the airflow parameter data comprising a single pair of two distinct airflow parameter values. (e.g., pressure and flow rate, impedance and flow rate) can be used to characterize different conduits or conduit-user interface combinations, and the characterization accuracy can be further improved by using two or more pairs of airflow parameter values from the laminar, transition, and turbulent regions of an airflow at low but increasing flow rate. At low flow rates, data measurement resolution and signal-to-noise can be further improved using a dithering process of collecting the airflow parameter values. For example, as further described below, ramp dithering can ramp the pressure, flow rate or speed of a motor generating the airflow. During such a ramp, multiple measurements of airflow parameter values (e.g., pressure, flow rate, impedance) can be taken and then averaged to improve accuracy. This improvement may also be achieved using, for example, the pressure or flow rate signal noise or injecting noise with a normal distribution.

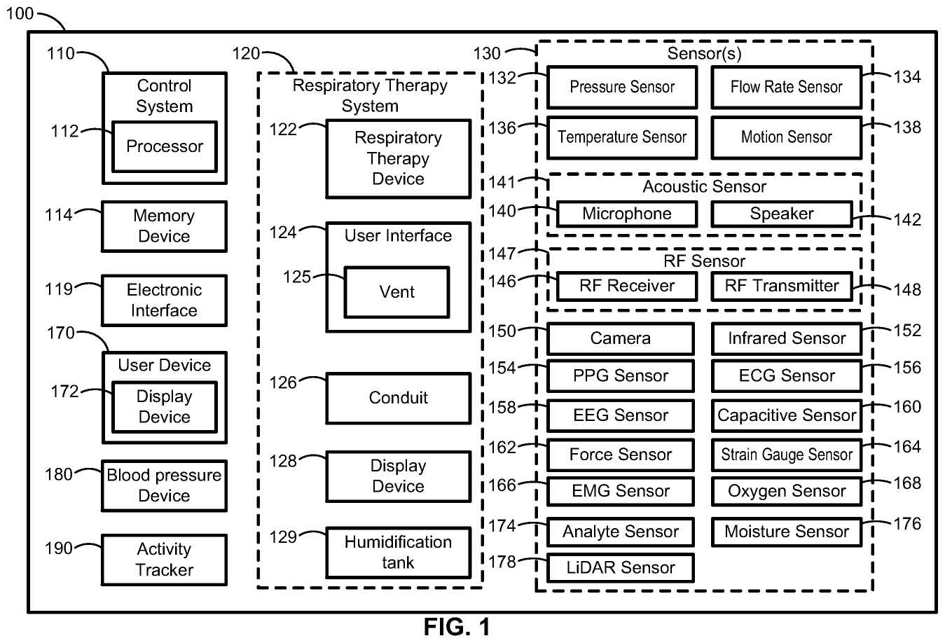

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory therapy system 120 (that includes a respiratory therapy device 122), a blood pressure device 180, an activity tracker 190, or any combination thereof.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with a user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120. The respiratory therapy system 120 can include a respiratory pressure therapy device (RPT) 122 (referred to herein as respiratory therapy device 122), a user interface 124 (also called a ‘mask’), a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance of the user's airways at a controlled target pressure that is nominally positive with respect to atmosphere throughout the user's respiratory cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 has a blower motor (not shown) that is generally used to generate pressurized air that is delivered to the user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 liters/minute and about 150 liters/minute, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of air at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

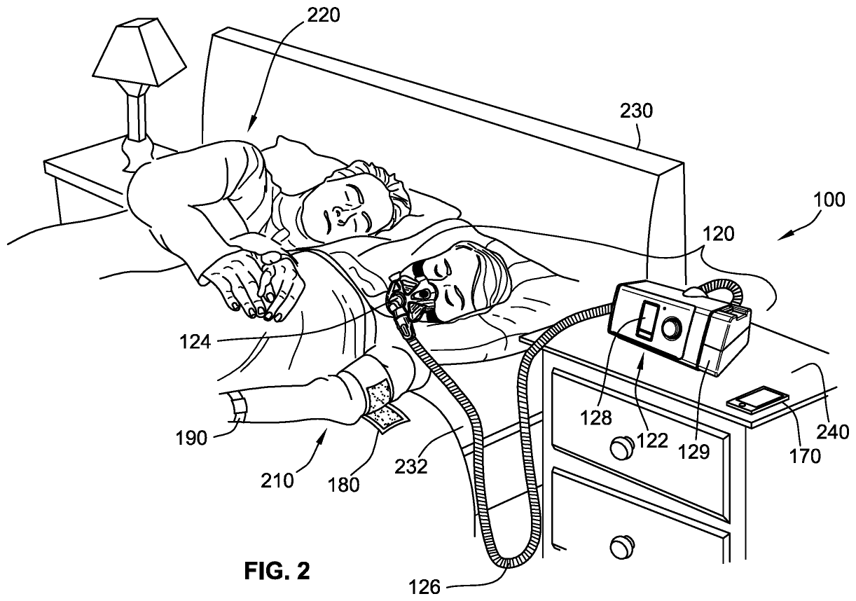

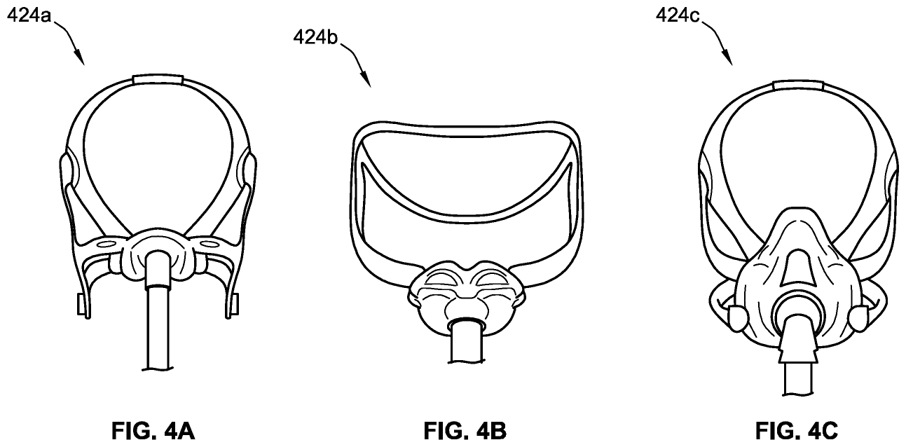

As shown in FIG. 2, in some implementations, the user interface 124 is a facial mask (e.g. a full face mask, also shown as 424c in FIG. 4C) that covers the nose and mouth of the user 210. Alternatively, the user interface 124 can be a nasal mask (shown as 424b in FIG. 4B) that provides air to the nose of the user 210 or a nasal pillow mask (shown as 424a in FIG. 4A) that delivers air directly to the nostrils of the user 210. The user interface 124 can include a plurality of straps forming, for example, a headgear for aiding in positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents 125 for permitting the escape of carbon dioxide and other gases exhaled by the user 210. In other implementations, the user interface 124 includes a mouthpiece (e.g., a night guard mouthpiece molded to conform to the teeth of the user 210, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit 126 for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130) described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score, also referred to as a myAir™ score, such as described in US 2017/0311879 A1, which is hereby incorporated by reference herein in its entirety; the current date/time; personal information for the user 210; questions seeking feedback from the user and/or advice to the user; etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include one or more vents (not shown) and a heater to heat the water in the humidification tank 129 in order to humidify the pressurized air provided to the user 210. Additionally, in some implementations, the conduit 126 can also include a heating element (e.g., coupled to and/or embedded in the conduit 126) that heats the pressurized air delivered to the user 210. The humidification tank 129 can be fluidly coupled to a water vapor inlet of the air pathway and deliver water vapor into the air pathway via the water vapor inlet, or can be formed in-line with the air pathway as part of the air pathway itself. In some implementations, the humidification tank 129 may not include the reservoir of water and thus waterless.

In some implementations, the system 100 can be used to deliver at least a portion of a substance from the receptacle (not shown) to the air pathway of the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined amount of pressurized air (e.g., determined by a sleep physician) to the user 210. The APAP system automatically varies the pressurized air delivered to the user 210 based on, for example, respiration data associated with the user 210. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring again to FIG. 2, a portion of the system 100 (FIG. 1), according to some implementations, is illustrated. The user 210 of the respiratory therapy system 120 and a bed partner 220) are located on a bed 230 and laying on a mattress 232. The user interface 124 (also referred to herein as a mask, e.g., a full face mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow rate signal, a pressure signal, respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, acoustic data, or both. Physiological data generated by one or more of the sensors 130 can be used by the control system 110 to determine a sleep-wake signal associated with the user 210 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, U.S. Pat. No. 10,492,720 B2, US 2014/0088373 A1, WO 2017/132726, WO 2019/122413, and US 2020/0383580 A1, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of and/or analyzed to determine (e.g., using the control system 110) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleep stage, an apnea-hypopnea index (AHI), pressure settings of the respiratory therapy device 122, or any combination thereof. The one or more events can include snoring, apneas, central apneas, obstructive apneas, mixed apneas, hypopneas, a mask leak (e.g., from the user interface 124), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 130, or from other types of data.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. Examples of flow rate sensors (such as, for example, the flow rate sensor 134) are described in U.S. Pat. No. 10,328,219 B2, which is hereby incorporated by reference herein in its entirety. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In one example, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user: for example, via a respiratory movement of the user. In some implementations, the motion data from the motion sensor 138 can be used in conjunction with additional data from another sensor 130 to determine the sleep state of the user.

The microphone 140 outputs sound and/or audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory therapy device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g., a sonar sensor), as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 122, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz. 18-22 KHz, or 17-18 kHz, for example), through the air. Such a system may be considered in relation to WO 2018/050913 and WO 2020/104465 mentioned above, each of which is hereby incorporated by reference herein in its entirety.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication can be Wi-Fi, Bluetooth, or the like.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230 (FIG. 2), and to determine a time when the user 210) exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a full face mask that covers the nose and mouth of the user 210, the analyte sensor 174 can be positioned within the full face mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the full face mask (in implementations where the user interface 124 is a full face mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 180, or any combination thereof. For example, the microphone 140 and the speaker 142 can be integrated in and/or coupled to the user device 170 and the pressure sensor 130 and/or flow rate sensor 132 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Home. Amazon Echo. Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 180 is generally used to aid in generating cardiovascular data for determining one or more blood pressure measurements associated with the user 210. The blood pressure device 180 can include at least one of the one or more sensors 130 to measure, for example, a systolic blood pressure component and/or a diastolic blood pressure component.

The activity tracker 190 is generally used to aid in generating physiological data for determining an activity measurement associated with the user 210. The activity tracker 190 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 190 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 190 is coupled (e.g., electronically or physically) to the user device 170.

In some implementations, the activity tracker 190 is a wearable device that can be worn by the user 210, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user 210. Alternatively, still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 180.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and optionally the user device 170. Thus, various systems can be formed using any portion or portions of the components shown and described herein and/or in combination with one or more other components.

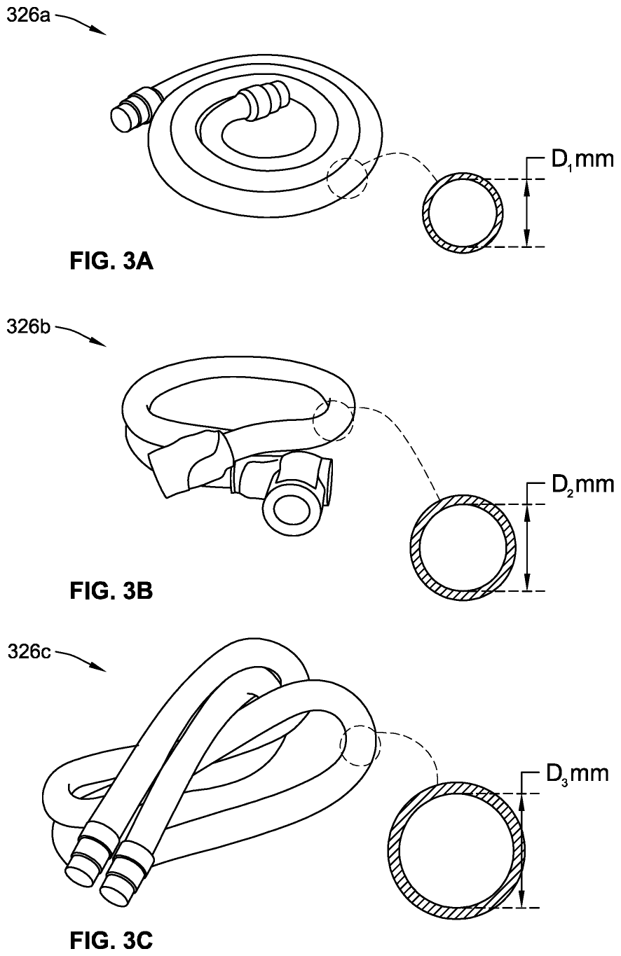

FIGS. 3A-3C illustrate perspective and cross-sectional views of conduits having different configurations. The illustrated conduits may have the same inner diameters, or may have different inner diameters and thus may be different types of conduits. FIG. 3A shows a conduit 326a having an inner diameter D₁ for use as the conduit 126 described above. FIG. 3B shows a conduit 326b having an inner diameter D₂ for use as the conduit 126 described above. FIG. 3C shows a conduit 326c having an inner diameter D; for use as the conduit 126 described above. In the implementations shown in FIGS. 3A-3B. D₁<D₂<D₃. In some non-limiting examples, D₁ is about 12 mm. D₂ is about 15 mm, and D₃ is about 19 mm. In other implementations D₁=D₂=D₃, and D₁ may be about 12 mm, or about 15 mm, or about 19 mm. In addition to variation in inner diameter, the conduits 326a, 326b, 326c in FIGS. 3A-3C may have different lengths such as, but not limited to, 1.8 meters, 2 meters, 3 meters, etc. During usage and testing, the conduits 326a, 326b, 326c in FIGS. 3A-3C may additionally have different configurations such as, but not limited to, one coils, two coils, three coils, four coils, a single bend, no bend, etc. Nevertheless, the methods and systems described herein can be implemented to characterize different conduits, optionally together with different user interfaces, irrespective of the configuration of the conduit. As defined herein. “characterizing” or “characterization of” the different conduits includes, among others, identifying presence or absence of the conduit, determining one or more dimensions of the conduit (e.g. length, inner diameter), determining a configuration of the conduit (e.g. number of coils), detecting presence or absence of a user interface coupled to the conduit, determining a type of user interface (e.g., full face mask, nasal mask, nasal pillow mask), determining presence or absence of an anti-asphyxia valve (AAV), determining a specific model of user interface (e.g., F10 full face mask by ResMed™, a P10 nasal pillows mask by ResMed™, a N20 nasal mask by ResMed™, other masks by ResMed™ or other manufacturers), or any combination thereof.

FIGS. 4A-4C illustrate perspective views of different types of user interfaces configured to be coupled to a conduit in the respiratory therapy system. FIG. 4A shows a nasal pillow mask 424a for use as the user interface 124 described above. The nasal pillow mask 424a is configured to deliver air directly to the nostrils of the user 210 and includes a cushion for comfortable positioning adjacent to the nose of the user 210. FIG. 4B shows a nasal mask 424b for use as the user interface 124 described above. The nasal mask 424b is configured to deliver air through the nose of the user 210 and includes straps for positioning on the head and face of the user 210. FIG. 4C shows a full face mask 424c for use as the user interface 124 described above. The full face mask 424c is configured to deliver air through the nose and the mouth of the user 210 and may include an anti-asphyxia valve (AAV). The AAV is a safety feature in full face masks wherein, if the respiratory therapy device stops delivering air for any reason (e.g., in a power failure), the AAV allows the user to breathe in fresh air from the environment rather than re-breathe exhaled air built up in the mask.

Any one of the user interfaces 424a. 424b. 424c shown in FIGS. 4A-4C as well as any other types of user interfaces, may be coupled to any one of the conduits shown in FIGS. 3A-3B or other types of conduits in any configuration for use during therapy. Accordingly, it is desirable to determine the type of conduit 126 and the user interface 124, such that they can be calibrated off-therapy (e.g., before or after therapy, before a first use) to deliver optimal amount of pressurized air to the user 210 during therapy. This is achieved through the systems and methods described herein, which characterize the conduit 126 coupled to the respiratory therapy device 122 in a respiratory therapy system 120 by determining a relationship between the conduit 126 and parameter data (e.g., pressure, flow rate, impedance, laminar flow constant, turbulent flow constant) of airflow through the conduit 126.

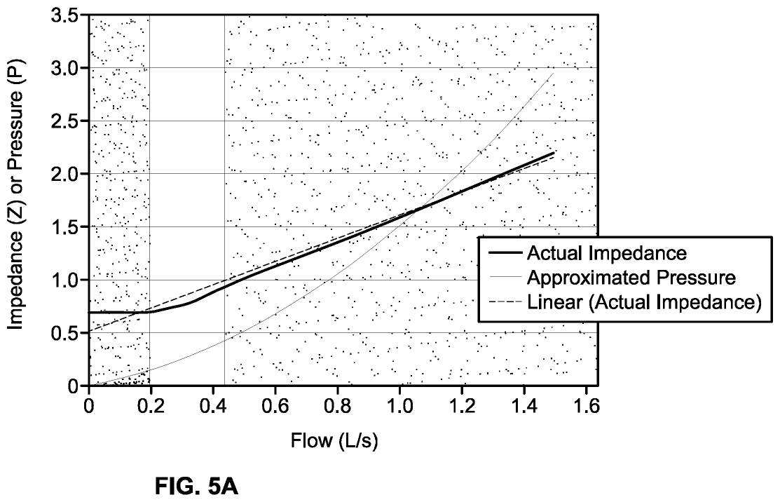

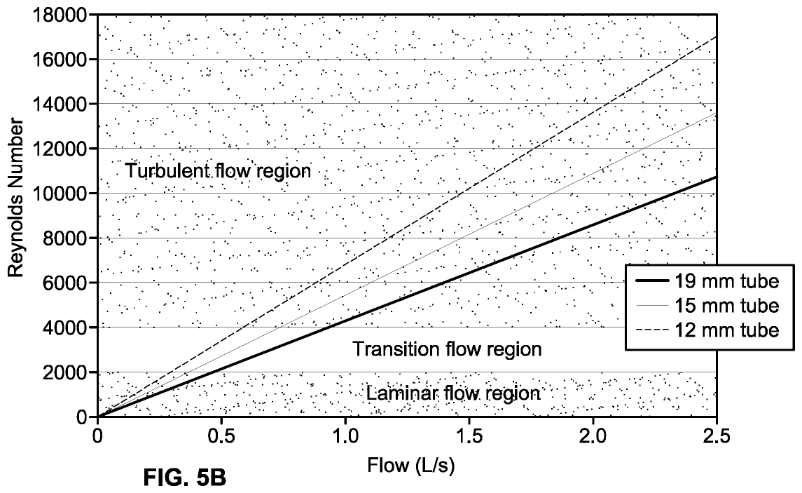

FIG. 5A illustrates a graphical representation of values of impedance (Z) or pressure (P) plotted over values of flow rate (Q) through the conduit 126 in the respiratory therapy system 120, while FIG. 5B illustrates a graphical representation of Reynolds number indicating laminar flow (Reynolds number between about 0-2300), transition flow (Reynolds number between about 2300-4000), and turbulent flow (Reynolds number higher than about 4000) plotted over values of flow rate corresponding to FIG. 5A.

As demonstrated by FIG. 5A, pressure P (measured in cm H₂O) approximately varies with flow rate Q (measured in L/s) as a continuous quadratic function passing through the origin (0,0) from the laminar flow region (flow rate between about 0-0.2 L/s) to the transition flow region (flow rate between about 0.2-0.45 L/s) and to the turbulent flow region (flow rate between about 0.45-1.6 L/s).

On the other hand, impedance Z (measured in cm H₂O/L/s) varies in a discrete nature with the flow rate Q (measured in L/s). The impedance is initially constant in the laminar flow region, curves upwards in the transition flow region, and then varies linearly with flow rate in the turbulent flow region. This can be approximated to a straight line, where the impedance Z is 0.5 cm H₂O/L/s at the start of flow and varies linearly with the flow rate Q.

FIG. 5B shows the different flow rates at which airflow through the conduit 126 having different inner diameters 12 mm. 15 mm, and 19 mm passes through the laminar flow region, transition flow region, and turbulent flow region and forms unique and distinguishable lines. As demonstrated by FIG. 5B, the Reynolds number varies linearly with flow rate of the airflow for a given conduit 126. It is contemplated that the flow rate values at which the airflow passes through the laminar flow region, transition flow region, and turbulent flow region would be modified by the effect of the humidification tank 129, and other components fluidly connected to the conduit 126. Nevertheless, it would be expected that the airflow through the conduit 126 having different inner diameters 12 mm, 15 mm, and 19 mm passing through the laminar flow region, transition flow region, and turbulent flow region at a flow rate of less than about 0.5 L/s would render unique and distinguishable lines, irrespective of the presence of a humidification tank 129 or other components fluidly connected to the conduit 126.

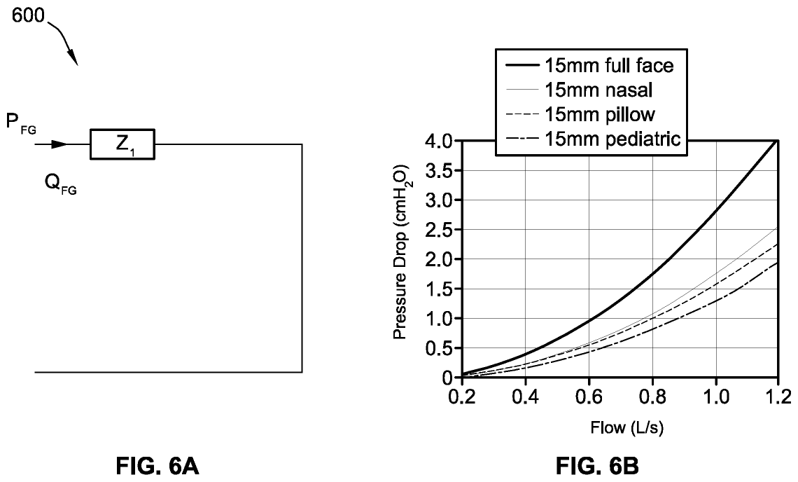

FIG. 6A shows a circuit diagram of an open airflow circuit 600 between the respiratory therapy device 122 and the conduit 126 in the respiratory therapy system 120. The open flow circuit 600 has an incoming airflow from the respiratory therapy device 122. The incoming airflow has pressure. P_{FG} and a flow rate, Q_{FG}, such that the input impedance is a ratio of pressure and flow rate, i.e. Zᵢₙ=(P_{FG}/Q_{FG}). The incoming airflow encounters a circuit impedance Z₁ representing the fluid resistance due to the conduit 126 or the combined fluid resistance due to the conduit 126 and the user interface 124, if connected to the conduit 126. The circuit impedance Z₁ causes a drop in pressure, dP, of the incoming airflow.

As described above, the respiratory therapy device 122 includes a pressure sensor 132 for measuring the pressure of the incoming airflow and a flow sensor 134 for measuring the flow rate of the incoming airflow from the respiratory therapy device 122. The drop in pressure dP due to the circuit impedance Z₁ is measured as the difference between the input pressure P_{FG} and pressure at the user interface 124, P_{M}, measured after the airflow crosses the circuit impedance Z₁, such that:

$$dP = {P}_{FG} - {P}_{M} = {Z}_{1} * {Q}_{FG}$$

Since combination of the conduit 126 and the user interface 124 opens into atmospheric pressure in the open airflow circuit 600, the pressure P_{M}=0, and hence:

$$dP = {P}_{FG} = {Z}_{1} * {Q}_{FG}$$

The variation in the pressure drop over the flow rate of airflow represents a unique and distinguishable signature identifying the combination of the conduit 126 and the user interface 124, if any, being used. FIG. 6B shows a graphical representation of variation of pressure drop (in cm H₂O) over flow rate (L/s) through four types of the user interface 124 (full face mask, nasal mask, nasal pillow mask, pediatric mask) coupled to the conduit 126 having an inner diameter of about 15 mm and placed in the open flow circuit 600 of FIG. 6A. The unique and distinguishable signature of the four types of user interface 124 takes the form of a quadratic function through the origin (0,0) of the graph, represented by:

$$dP = {P}_{FG} = {K}_{1} * {Q}_{FG}^{2} + {K}_{2} * {Q}_{FG} ,$$

where K₁, K₂ are coefficients of the flow rate Q_{FG}, and unique for the combination of the conduit 126 and the user interface 124, if any, being used. Thus, the coefficients K₁, K₂ represent the unique and distinguishable signature of the combination of the conduit 126 and the user interface 124, if any, being used.

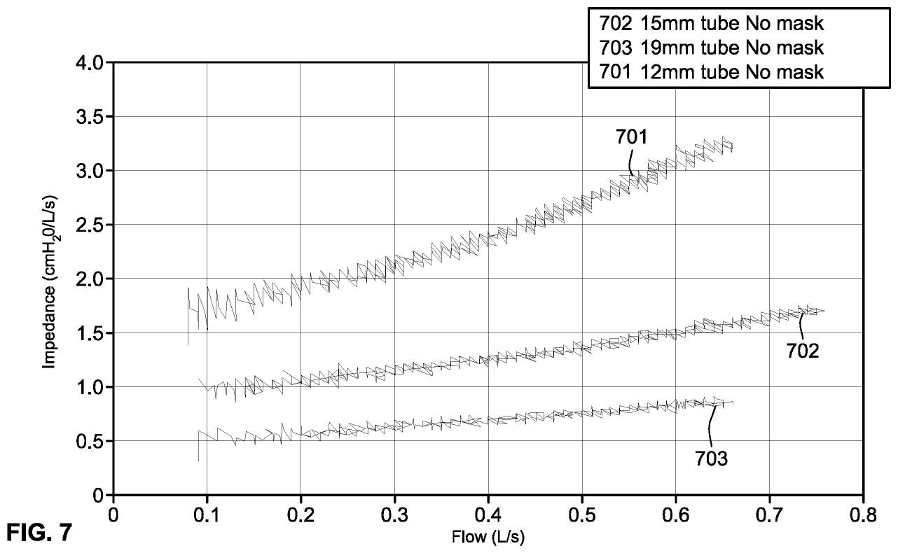

FIG. 7 illustrates a graphical representation of impedance (in cm H₂O/L/s) over flow rate (L/s) through three different types of conduit 126 that are not coupled to the user interface 124 in the respiratory therapy system 120. The three different types of the conduit 126 are (i) a tube having an inner diameter 15 mm represented by a trace 701, (ii) a tube having an inner diameter 12 mm represented by a trace 702, and (iii) a tube having an inner diameter 19 mm represented by a trace 703. FIG. 7 demonstrates based on the airflow parameter data—impedance over flow rate, in this case—that each of the three different types of conduit 126 carry a unique and distinguishable signature that helps characterize the conduit 126.

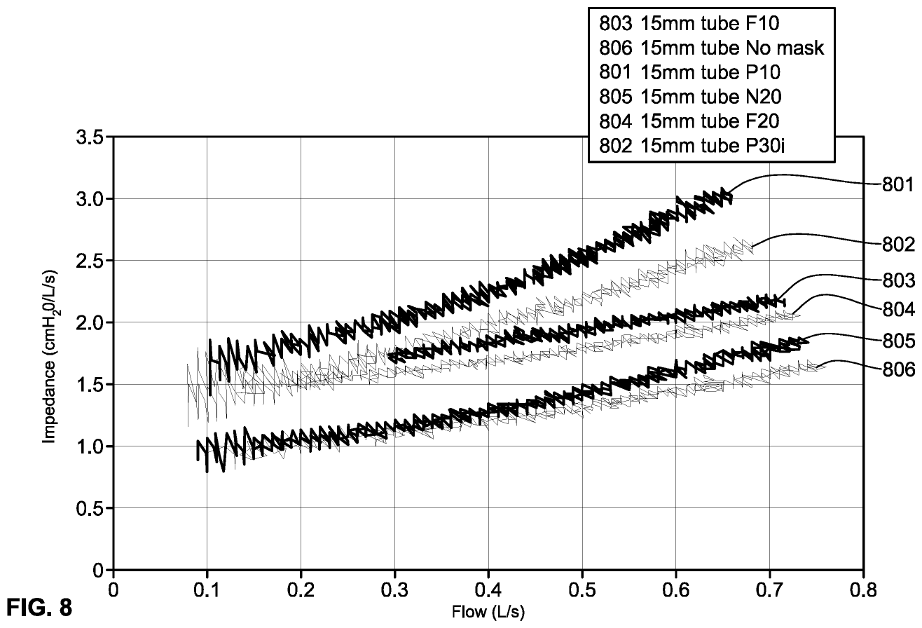

FIG. 8 illustrates a graphical representation of impedance (in cm H₂O/L/s) over flow rate (L/s) through five different types of mask, e.g., user interfaces 124, coupled to a tube, e.g., conduit 126 of the respiratory therapy system 120, having an inner diameter of about 15 mm. The five different types of user interfaces 124 tested are (i) a F10 full face mask by ResMed™ represented by a trace 803, (ii) a P10 nasal pillows mask by ResMed™ represented by a trace 801, (iii) a N20 nasal mask by ResMed™ represented by a trace 805, (iv) a F20 full face mask by ResMed™ represented by a trace 804, and (v) a P30i nasal pillows mask by ResMed™ represented by a trace 802. A graph of impedance (in cm H₂O/L/s) over flow rate (L/s) through the conduit 126 of inner diameter 15 mm with no mask coupled thereto is also depicted in a trace 806 for comparison. FIG. 8 demonstrates based on the airflow parameter data—impedance over flow rate, in this case—that each of the five different types of user interface 124 as well as only the conduit 126 of inner diameter of about 15 mm in the absence of the user interface 124 carries a unique and distinguishable signature that helps characterize the user interface 124.

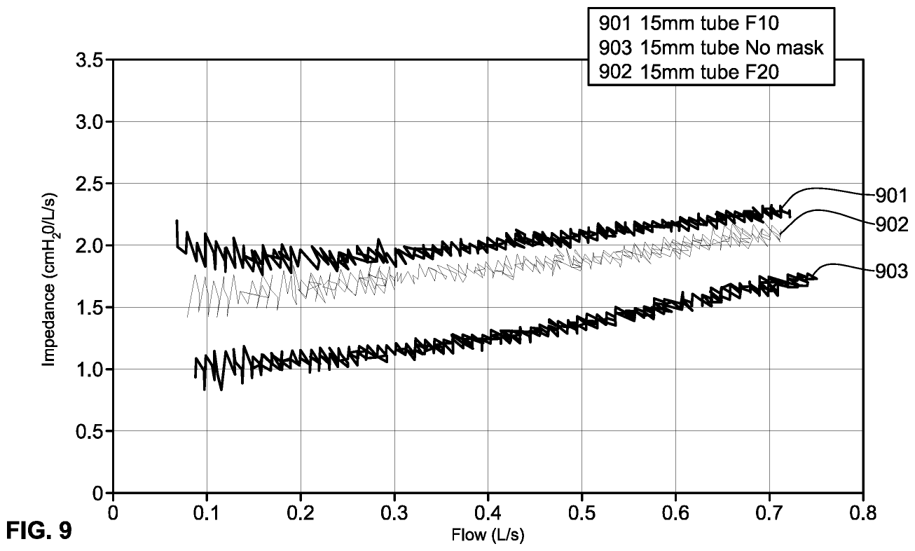

FIG. 9 illustrates a graphical representation of impedance (in cm H₂O/L/s) over flow rate (L/s) through two different types of user interfaces 124 coupled to the conduit 126 having an inner diameter of about 15 mm in the respiratory therapy system 120, wherein the user interface 124 includes an anti-asphyxia valve (AAV). The two different types of user interfaces 124 are (i) a F10 full face mask by ResMed™ having an AAV represented by a trace 901, and (ii) a F20 full face mask by ResMed™ having an AAV representing a trace 902. A graph of impedance (in cm H₂O/L/s) over flow rate (L/s) through the conduit 126 of inner diameter 15 mm with no mask coupled thereto is also depicted as a trace 903 for comparison. FIG. 9 demonstrates based on the airflow parameter data—impedance over flow rate, in this case—that the AAV in the user interface 124 contributes to a unique and distinguishable signature that can help characterize the conduit 126 coupled to the user interface 124 having an AAV. As an example, a characteristic “bend” (non-linear portion) in the illustrated graph of F10 full face mask at flow rates of between about 0.2 L/s and 0.3 L/s indicates presence of the AAV.

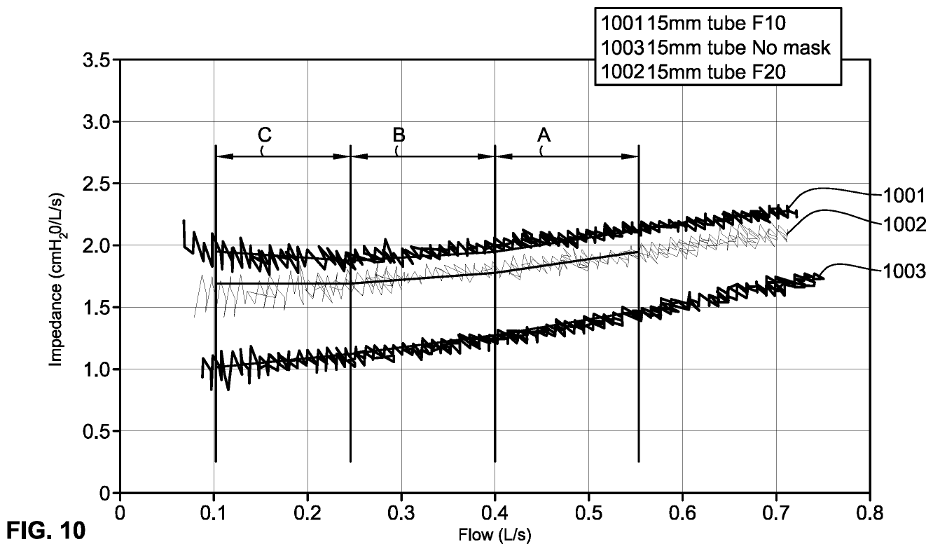

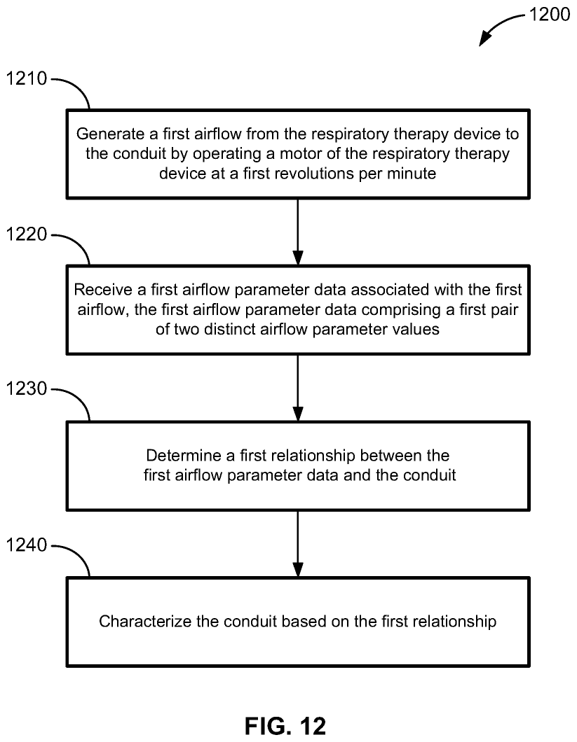

FIG. 10 illustrates improved resolution of airflow parameter data in FIG. 9 due to airflow parameter data collected in epochs A, B. C across different regions of the flow spectrum from laminar flow to turbulent flow. The two different types of user interfaces 124 are (i) a F10 full face mask by ResMed™ having an AAV represented by a trace 1001, and (ii) a F20 full face mask by ResMed™ having an AAV representing a trace 1002. A trace 1003 of impedance (in cm H₂O/L/s) over flow rate (L/s) through the conduit 126 of inner diameter 15 mm with no mask coupled thereto is also provided for comparison. The epochs A, B, and C shown in FIG. 10 could occupy zones of similar or different intervals of flow rate ranges extending across regions of laminar flow (Reynolds number between about 0-2300), transition flow (Reynolds number between about 2300-4000), and turbulent flow (Reynolds number higher than about 4000). Airflow parameter values such as, but not limited to, pressure P and impedance Z are measured in one or more of the epochs A, B, and C. The method 1200, described below with respect to FIG. 12, is then used to determine a relationship between the airflow parameter data having the airflow parameter values, and particular combinations of the conduit 126 and the user interface 124. In non-limiting implementations, determining the relationship may include estimating a measure of central tendency of each type of airflow parameter value (e.g., pressure, impedance, flow rate) within these epochs A, B, and C, and/or estimating a slope between points representing the collected values (e.g., pressure P over flow rate Q, impedance Z over flow rate Q) or the measure of central tendency of the collected values (e.g., average pressure P over average flow rate Q within one or more of the epochs A, B, or C). As non-limiting examples, curves plotting pressure P over flow rate Q are typically quadratic in nature, while curves plotting impedance Z over flow rate Q are typically linear in nature, except for airflow parameter data collected in the transition region which may be represented as a non-linear portion of the curve.

Overall. FIG. 10 demonstrates that collection of airflow parameter data carries a unique and distinguishable signature for any combination of the conduit 126 and the user interface 124, regardless of whether the airflow through the conduit 126 and the user interface 124 falls in the laminar flow region, the transition flow region, and/or the turbulent flow region. Impedance characteristics from a combination of the laminar flow region, the transition flow region, and/or the turbulent flow region contain unique and distinguishable signatures, which allows further improved classification of the airflow parameter data.

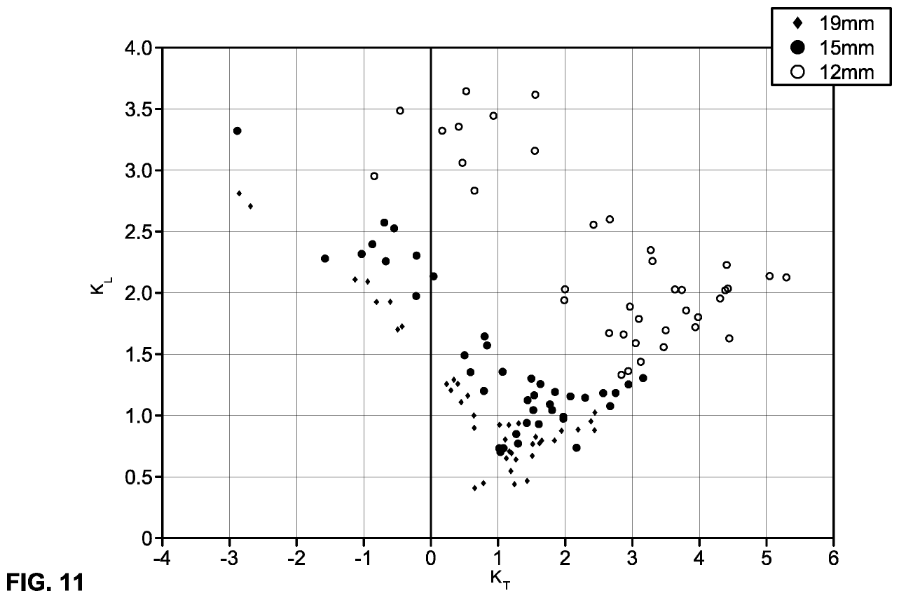

FIG. 11 illustrates a graphical representation of the values of a laminar flow constant, K_{L}, over values of turbulent flow constant, K_{T}, measured for different configurations of the conduit 126 with or without being coupled to the user interface 124 in the respiratory therapy system 120. The different configurations include combinations of three different types of the conduit 126 having inner diameters 12 mm, 15 mm, and 19 mm—in straight, bent, or coiled configuration—coupled to different types of user interfaces (e.g. different models of full face mask, nasal mask, nasal pillows mask, etc.). Each point plotted on the graph represents a unique conduit (e.g., 12 mm, 15 mm, and 19 mm) or a unique combination of conduit and user interface. The laminar flow constant, K_{L}, and the turbulent flow constant, K_{T}, are linearly related in the open flow circuit 600 shown in FIG. 6A, where the circuit impedance Z₁ is expressed as a ratio of the pressure P_{FG} and the flow rate Q_{FG}:

$${Z}_{1} = {Z}_{in} = {P}_{FG} / {Q}_{FG} = {K}_{T} * {Q}_{FG} + {K}_{L} .$$

For any two distinct measurements of pressure P_{FG} and the flow rate Q_{FG}, the laminar flow constant, K_{L}, and the turbulent flow constant, K_{T} can be determined and represents a unique and distinguishable signature of the combination of the conduit 126 and the user interface 124 forming the circuit impedance Z₁. Accordingly, the different combinations of the conduit 126 and the user interface 124 forming the circuit impedance Z₁ can be represented by a unique point when K_{T} and K_{L} are plotted. This helps identify the combination of the conduit 126 and the user interface 124 being used.

The graphical representations demonstrated by FIGS. 7-11 are developed using methods of characterizing the conduit 126 coupled to the respiratory therapy device 122 in the respiratory therapy system 120. FIG. 12 illustrates a flow diagram for such a method 1200 of characterizing a conduit. Referring to the flow diagram, at step 1210, a first airflow is generated from a respiratory therapy device (e.g. the respiratory therapy device 122) to a conduit (e.g., the conduit 126) by operating a motor of the respiratory therapy device at a first revolutions per minute (RPM). In some implementations, the motor of the respiratory therapy device may operate between about 3,000 RPM and about 12,000 RPM. As noted above, the conduit may have different dimensions and configurations such as, but not limited to, inner diameters of 12 mm, 15 mm, 19 mm, lengths of 1.8 meters, 2 meters, 3 meters, having one coil, having two coils, having three coils, having four coils, connected to a full face mask, connected to a nasal mask, connected to a nasal pillows mask, etc.

The first airflow maybe generated by operating the motor of the respiratory therapy device at particular motor speeds between about 3,000-12,000 RPM, or averaged across a range of motor speeds within that range (e.g. 3,000-4,000 RPM, 4,000-5,000 RPM, 5,000-6,000 RPM). The first airflow may be generated for between about 2 seconds and 5 seconds. For example, the first airflow maybe generated for at least about 2 seconds, at least about 3 seconds, at least about 4 seconds, at least about 5 seconds, etc. Additionally, or alternatively, the first airflow maybe generated for not more than about 30 seconds, not more than about 20 seconds, not more than about 10) seconds, not more than about 5 seconds, not more than about 2 seconds, not more than about 1 second, etc. The first airflow maybe generated in the laminar flow region with Reynolds number between about 0 and 2300, in the transition region with Reynolds number between about 2300 and 4000, and/or in the turbulent region with Reynolds number higher than about 2300. In some implementations, the first airflow may have a flow rate of between about 0.1-0.8 liters per second (L/s). In non-limiting examples, the first airflow may have a flow rate of about 0.2 L/s, about 0.3 L/s, about 0.4 L/s, about 0.5 L/s, about 0.6 L/s, about 0.7 L/s, between about 0.2-0.4 L/s, between about 0.3-0.5 L/s, between about 0.4-0.6 L/s, between about 0.5-0.7 L/s, no more than 0.3 L/s, no more than 0.4 L/s, no more than 0.5 L/s, no more than 0.6 L/s, no more than 0.7 L/s, etc. In some implementations, the first airflow may have an associated noise level of no more than about 40 dBA measured at a distance of about one meter from the respiratory therapy device. As is well-known in the art, sound expressed in dBA indicates a relative loudness of sound in air as perceived by the human ear since the human ear is less sensitive at low audio frequencies, especially below 1000 Hz, than at high audio frequencies.

In particular, the first airflow parameter data generated in the transition region offers the benefits of improved resolution to clearly characterize the conduit without generating noise from the motor of the respiratory therapy device. Further, in implementations where the user interface includes an anti-asphyxia valve (AAV), the first airflow may have a flow rate value of less than about 0.4 liters per second, since the AAV may open at the flow rate value greater than 0.4 liters per second.

At step 1220, a first airflow parameter data associated with the first airflow is received. The first airflow parameter data includes a first pair of airflow parameter values. The first pair of airflow parameter values comprises two distinct airflow parameter values. In some implementations, the first airflow parameter data includes a plurality of first pairs of airflow parameter values associated with the first airflow. The plurality of first pairs of airflow parameter values associated with the first airflow may be collected using a dithering process, which improves both the resolution of the airflow parameter data as well as the signal-to-noise ratio. During the dithering process, the motor of the respiratory therapy device is operated with gradually increasing or decreasing RPMs within a range and the airflow parameter values (e.g., pressure, flow rate, impedance) for each RPM are measured.

In some implementations, the first pair of airflow parameter values includes any two of a pressure value, a flow rate value, and an impedance value calculated as a ratio of the pressure value and the flow rate value of the first airflow. In other implementations, the first pair of airflow parameter values includes a laminar flow constant (e.g., the laminar flow constant K_{L} described above) and a turbulent flow constant (e.g., the turbulent flow constant K_{T} described above).

The first airflow parameter data is generated by one or more sensors (e.g., sensors 130) communicatively coupled to the respiratory therapy system. For example, a flow rate value of the first airflow is generated by a flow sensor (e.g., flow sensor 134) communicatively coupled to the respiratory therapy device. As another example, a pressure value of the first airflow is generated by a pressure sensor (e.g., pressure sensor 132) communicatively coupled to the respiratory therapy device.

At step 1230, a first relationship between the first airflow parameter data and the conduit is determined. In some implementations where the first pair of airflow parameter values includes a laminar flow constant and a turbulent flow constant, the first relationship is a linear relationship for a given pressure value and a given flow rate of the first airflow (see FIG. 11 and associated description above).

In some implementations, where the first pair of airflow parameter values includes any two of a pressure value, a flow rate value, and an impedance value, and where the first airflow parameter data includes a plurality of first pairs of airflow parameter values associated with the first airflow, determining the first relationship may include estimating a first measure of central tendency associated with the first airflow parameter data. This comprises computing the measure of central tendency independently for each type of airflow parameter value (e.g. pressure values, flow rate values, impedance values) generated by the dithering process discussed with respect to step 1220 above. The first measure of central tendency may be a mean, a median, or a mode of the first airflow parameter data. In some implementations, the first measure of central tendency is estimated by excluding any airflow parameter value that is at least two standard deviations away from the mean of the first airflow parameter data.

At step 1240, the conduit is characterized based on the first relationship. As described above, characterizing the conduit may include determining, among other things, presence or absence of the conduit, a type of the conduit, presence or absence of a user interface coupled to the conduit, determining a type of the user interface, and the like. In some implementations, a specific model of user interface (e.g., F10 full face mask by ResMed™, a P10 nasal pillows mask by ResMed™, a N20 nasal mask by ResMed™) coupled to the conduit may be identified. Characterizing the conduit may be implemented using a classification-based machine learning algorithm using training data labeled with characterizations of the conduit, as further discussed below:

In some implementations where determining the first relationship includes estimating the first measure of central tendency associated with the first airflow parameter data, characterizing the conduit is based on the first measure of central tendency satisfying a first condition. Satisfying the first condition may include exceeding a first threshold value, not exceeding the first threshold value, staying within a first predetermined range of values, staying outside the first predetermined range of values, or any combination thereof. In such implementations, both the first threshold value and the first predetermined range of values are associated with a pressure value, a flow rate value, and an impedance value determined at the first RPM, which may be used as training data for training the classification-based machine learning algorithm.

In some implementations, the method 1200 may further include generating a second airflow from the respiratory therapy device to the conduit by operating the motor of the respiratory therapy device at a second RPM and receiving a second airflow parameter data associated with the second airflow. The second airflow parameter data is generated by one or more sensors communicatively coupled to the respiratory therapy system and typically the same sensor(s) from which the first airflow parameter data are generated. For example, a flow rate value of the second airflow is generated by a flow sensor communicatively coupled to the respiratory therapy device. As another example, a pressure value of the first airflow is generated by a pressure sensor communicatively coupled to the respiratory therapy device.

The second airflow parameter data includes a second pair of airflow parameter values. The second pair of airflow parameter values comprises two distinct airflow parameter values. In some implementations, the second airflow parameter data includes a plurality of second pairs of airflow parameter values associated with the second airflow. The plurality of second pairs of airflow parameter values associated with the second airflow may be collected using the dithering process described above at step 1220. In some implementations, the second pair of airflow parameter values includes any two of a pressure value, a flow rate value, and an impedance value calculated as a ratio of the pressure value and the flow rate value of the second airflow: In other implementations, the second pair of airflow parameter values includes a laminar flow constant and a turbulent flow constant.

The second airflow maybe generated by operating the motor of the respiratory therapy device at particular motor speeds between about 3,000-12,000 RPM, or averaged across a range of motor speeds within that range (e.g., 3,000-4,000 RPM, 4,000-5,000 RPM, 5,000-6,000 RPM). The second airflow may be generated for between about 2 seconds and 5 seconds. For example, the second airflow maybe generated for at least about 2 seconds, at least about 3 seconds, at least about 4 seconds, at least about 5 seconds. Additionally, or alternatively, the second airflow maybe generated for not more than about 30 seconds, not more than about 20 seconds, not more than about 10) seconds, not more than about 5 seconds, not more than about 2 seconds, not more than about 1 second, etc. The second airflow maybe generated in the laminar flow region with Reynolds number between about 0 and 2300, in the transition region with Reynolds number between about 2300 and 4000, and/or in the turbulent region with Reynolds number higher than about 4000. In some implementations, the second airflow may have a flow rate of between about 0.1-0.8 liters per second (L/s). In non-limiting examples, the second airflow may have a flow rate of about 0.2 L/s, about 0.3 L/s, about 0.4 L/s, about 0.5 L/s, about 0.6 L/s, about 0.7 L/s, between about 0.2-0.4 L/s, between about 0.3-0.5 L/s, between about 0.4-0.6 L/s, between about 0.5-0.7 L/s, no more than 0.3 L/s, no more than 0.4 L/s, no more than 0.5 L/s, no more than 0.6 L/s, no more than 0.7 L/s, etc. In some implementations, the second airflow may have an associated noise level of no more than about 40 dBA measured at a distance of about one meter from the respiratory therapy device.

In particular, the second airflow parameter data generated in the transition region offers the benefits of improved resolution to clearly characterize the conduit without generating noise from the motor of the respiratory therapy device. Further, in implementations where the user interface includes an anti-asphyxia valve (AAV), the second airflow may have a flow rate value of less than about 0.4 liters per second, since the AAV may open at the flow rate value greater than 0.4 liters per second.

Subsequent to receiving the second airflow parameter data, a second relationship between the second airflow parameter data and the conduit is determined. In some implementations where the second pair of airflow parameter values includes a laminar flow constant and a turbulent flow constant, the second relationship is a linear relationship for a given pressure value and a given flow rate of the second airflow.

In some implementations, where the second pair of airflow parameter values includes any two of a pressure value, a flow rate value, and an impedance value, and where the second airflow parameter data includes a plurality of second pairs of airflow parameter values associated with the second airflow, determining the second relationship may include estimating a second measure of central tendency associated with the second airflow parameter data. This comprises computing the measure of central tendency independently for each type of airflow parameter value (e.g. pressure values, flow rate values, impedance values) generated by the dithering process described above. The second measure of central tendency may be a mean, a median, or a mode of the second airflow parameter data. In some implementations, the second measure of central tendency is estimated by excluding any airflow parameter value that is at least two standard deviations away from the mean of the second airflow parameter data.

The conduit can then be characterized based on the second relationship. Characterizing the conduit may be implemented using a classification-based machine learning algorithm using training data labeled with characterizations of the conduit, as further discussed below.

In some implementations where determining the second relationship includes estimating the second measure of central tendency associated with the second airflow parameter data, characterizing the conduit is based on the second measure of central tendency satisfying a second condition. Satisfying the second condition may include exceeding a second threshold value, not exceeding the second threshold value, staying within a second predetermined range of values, staying outside the second predetermined range of values, or any combination thereof. In such implementations, both the second threshold value and the second predetermined range of values are associated with a pressure value, a flow rate value, and an impedance value determined at the second RPM and used as training data for training the classification-based machine learning algorithm.

In some implementations, the method 1200 may characterize the conduit based on the first pair (or plurality of first pairs) of airflow parameter values associated with the first airflow and the second pair (or plurality of second pairs) of airflow parameter values associated with the second airflow. In a non-limiting example, the method 1200 may estimate a first slope of a line through each pair of airflow parameter values associated with the first airflow and each pair of airflow parameter values associated with the second airflow. In such implementations, characterizing the conduit is based on the first slope satisfying a third condition.

In another non-limiting example, the method 1200 may estimate a second slope of a line through the first measure of central tendency associated with the first airflow parameter data and the second measure of central tendency associated with the second airflow parameter data. In such implementations, characterizing the conduit is based on the second slope satisfying the third condition. Satisfying the third condition may include exceeding a third threshold value, not exceeding the third threshold value, staying within a third predetermined range of values, staying outside the third predetermined range of values, or any combination thereof. In such implementations, both the third threshold value and the third predetermined range of values are used as training data for training the classification-based machine learning algorithm.

Based on the characterization of the conduit and the user interface coupled thereto using the above-described method, a third airflow parameter value can be determined for prospective delivery of pressurized airflow to a user, when the user wears the user interface for respiratory therapy. Such determination of the third airflow parameter value would include determining impedance due to intentional leak through one or more vents in the user interface (described above), unintentional leak at the user interface or a mouth of the user, or a combination of both.

The method 1200 may include generating a plurality of airflows in one or more of the laminar flow region, the transition flow region, and the turbulent flow region by operating the motor at various RPMs and receiving a plurality of airflow parameter data associated therewith. The airflow parameter data may include pairs of airflow parameter values (e.g., flow rate, pressure, impedance, etc.) that either represent the single values or the measures of central tendency (e.g., mean, median, mode) of the airflow parameter data collected by operating the motor in the respiratory therapy device at different RPMs, in different flow regimes (e.g. laminar flow region, transition flow region, turbulent flow region) or epochs comprising flow rate ranges that overlap with one or more of the different flow regimes (see FIG. 10). The plurality of airflow parameter data may be analyzed to determine a relationship with the conduit for accurately characterizing the conduit.

The method 1200 can be implemented using any classification-based supervised or unsupervised machine learning algorithm using training data labeled with characterizations of the conduit. The algorithm may be trained using airflow parameter data indicating unique and distinguishable signatures of the different combinations of conduits and user interfaces coupled thereto forming the different configurations (e.g., as shown in FIGS. 7-11).

In some specific implementations, the classification-based machine learning algorithm may use a KNN (K-Nearest Neighbor) technique by classifying based on an Euclidean distance of the airflow parameter value from the trained data classifying the characterization of the conduit. As a non-limiting example, the KNN technique uses a linear regression curve fitting of the airflow parameters values such as the laminar flow constant K_{L} and the turbulent flow constant, K_{T}, described above.

In other implementations, the classification-based machine learning algorithm may utilize more basic machine learning tools including decision trees (“DT”), Bayesian networks (“BN”), artificial neural network (“ANN”), or support vector machines (“SVM”). In other examples, deep learning algorithms or other more sophisticated machine learning algorithms, e.g., convolutional neural networks (“CNN”), recurrent neural networks (“RNN”), or capsule networks (“CapsNet”) may be used.

DT are classification graphs that match user input data to device data at each consecutive step in a decision tree. The DT program moves down the “branches” of the tree based on the user input to the recommended device settings.

Bayesian networks (“BN”) are based on a likelihood something is true based on given independent variables and are modeled based on probabilistic relationships. BN are based purely on probabilistic relationships that determine the likelihood of one variable based on another or others.

Artificial neural networks (“ANN”) are computational models inspired by an animal's central nervous system. They map inputs to outputs through a network of nodes. However, unlike BN, in ANN the nodes do not necessarily represent any actual variable. Accordingly, ANN may have a hidden layer of nodes that are not represented by a known variable to an observer. ANNs are capable of pattern recognition. Their computing methods make it easier to understand a complex and unclear process that might go on during determining a symptom severity indicator based a variety of input data.

Support vector machines (“SVM”) came about from a framework utilizing of machine learning statistics and vector spaces (linear algebra concept that signifies the number of dimensions in linear space) equipped with some kind of limit-related structure. In some cases, they may determine a new coordinate system that easily separates inputs into two classifications. For example, a SVM could identify a line that separates two sets of points originating from different classifications of events.

Deep neural networks (DNN) have developed recently and are capable of modeling very complex relationships that have a lot of variation. Various architectures of DNN have been proposed to tackle the problems associated with algorithms such as ANN by many researchers during the last few decades. These types of DNN are CNN (Convolutional Neural Network), RBM (Restricted Boltzmann Machine), LSTM (Long Short Term Memory) etc. They are all based on the theory of ANN. They demonstrate a better performance by overcoming the back-propagation error diminishing problem associated with ANN.

In yet other implementations, the classification-based machine learning algorithm may use linear regression, logistic regression, gradient descent, random forest, quadratic discriminant analysis, or any combination of the above techniques.

Machine learning models require training data to identify the features of interest that they are designed to detect. For instance, various methods may be utilized to form the machine learning models, including applying randomly assigned initial weights for the network and applying gradient descent using back propagation for deep learning algorithms. In other examples, a neural network with one or two hidden layers can be used without training using this technique.

According to some implementations of the present disclosure, the output characterizing the conduit is associated with a confidence interval that such characterization is correct. In some implementations, such characterization of the conduit maybe compared against a characterization of the conduit obtained through an acoustic signature of airflow through the conduit to determine how closely the characterization of the conduit through the latter method and the method 1200 are aligned.

Generally, the method 1200 can be implemented using a system having a control system with one or more processors, and a memory storing machine readable instructions. The controls system can be coupled to the memory, and the method 1200 can be implemented when the machine readable instructions are executed by at least one of the processors of the control system. The method 1200 can also be implemented using a computer program product (such as a non-transitory computer readable medium) comprising instructions that when executed by a computer, cause the computer to carry out the steps of the method 1200.

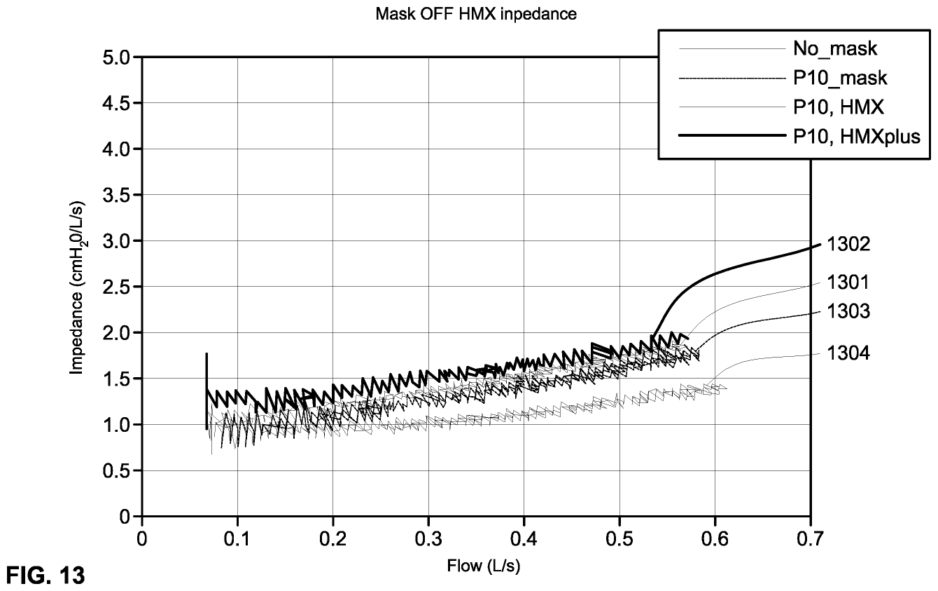

Information sensed through the conduit may also allow detection of the presence and identification of a waterless humidifier in a respiratory therapy system. FIG. 13 illustrates a graphical representation of impedance (in cm H₂O/L/s) over flow rate (L/s) through a type of user interface 124 coupled to the conduit 126 in the respiratory therapy system 120, wherein respiratory therapy system 120 may include a waterless humidifier. In this example, the type of user interface 124 is a P10 nasal pillows mask by ResMed™ and the user interface was not worn on the face during testing. A trace 1301 shows the impedance (in cm H₂O/L/s) over flow rate (L/s) through the conduit 126 with a first type of waterless humidifier and a trace 1302 shows the impedance through the conduit 126 with a second type of waterless humidifier. A trace 1303 of impedance (in cm H₂O/L/s) over flow rate (L/s) through the conduit 126 with only the P10 mask without a waterless humidifier and a trace 1304 of no mask coupled to the conduit 126 is also depicted for comparison. In this example, the inner diameter of the conduit 126 is the same for all the instances represented in FIG. 13.

In this example, the first type of waterless humidifier is a HumidX type waterless humidifier while the second type of waterless humidifier is a HumidX Plus type waterless humidifier, both available from ResMed. The HumidX™ and HumidX Plus™ waterless humidifiers are small heat and moisture exchangers that may be located in the air pathway between the respiratory therapy device and user interface providing effective humidification for users in most climates and sleep environments. The HumidX™ type is a standard waterless humidification system, suitable for most climates. The HumidX Plus™ type is intended for use in dry and high altitude environments where humidity is low (e.g. airplane cabins) and has higher capacity for capturing more moisture and heat during exhalation and returning to the airways upon inspiration.

FIG. 13 demonstrates based on the airflow parameter data—impedance over flow rate, in this case—that the presence as well as the type of waterless humidifier used with the user interface 124 contributes to a unique and distinguishable signature that can help determine the presence as well as the type of waterless humidifier used with the user interface 124. In this example, the k1 and k2 values in respect of the type of waterless humidifier are calculated from the flow and impedance data obtained from sensors as described above, and the conduit/mask/waterless humidifier combinations are identified as described in relation to the tube/mask/AAV combinations described above.

One or more elements or aspects or steps, or any portion(s) thereof, from one or more of any of claims 1-100 below can be combined with one or more elements or aspects or steps, or any portion(s) thereof, from one or more of any of the other claims 1-100 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for characterizing a conduit coupled to a respiratory therapy device in a respiratory therapy system, the method comprising:
generating a first airflow from the respiratory therapy device to the conduit by operating a motor of the respiratory therapy device at a first revolutions per minute;
receiving a first airflow parameter data associated with the first airflow, the first airflow parameter data comprising a first pair of two distinct airflow parameter values;
determining a first relationship between the first airflow parameter data and the conduit; and
characterizing the conduit based on the first relationship.

2. The method of claim 1, wherein the characterizing the conduit comprises determining (i) presence or absence of the conduit, (ii) a type of the conduit having a specific inner diameter, (iii) presence or absence of a user interface coupled to the conduit, (iv) determining a type of the user interface coupled to the conduit, or (v) any combination thereof.

3-5. (canceled)

6. The method of claim 1, wherein the type of user interface is one of (i) a full face mask, (ii) a nasal mask, or (iii) a nasal pillows mask.

7. The method of claim 1, wherein the first airflow parameter data comprises a plurality of first pairs of airflow parameter values associated with the first airflow.

8. The method of claim 1, wherein the first pair of airflow parameter values includes any two of: (i) a pressure value, (ii) a flow rate value, (iii) an impedance value calculated as a ratio of the pressure value and the flow rate value of the first airflow, (iv) a laminar flow constant, and (v) a turbulent flow constant.

9-10. (canceled)

11. The method of claim 1, wherein the determining the first relationship further comprises:
estimating a first measure of central tendency associated with the first airflow parameter data; and
characterizing the conduit based on the first measure of central tendency associated with the first airflow parameter data satisfying a first condition.

12-16. (canceled)

17. The method of claim 1, wherein the first airflow is generated for not more than 5 seconds.

18. The method of claim 1, further comprising:
generating a second airflow from the respiratory therapy device to the conduit by operating the motor of the respiratory therapy device at a second revolutions per minute;
receiving a second airflow parameter data associated with the second airflow, the second airflow parameter data comprising a second pair of two distinct airflow parameter values; and
determining a second relationship between the second airflow parameter data and the conduit.

19-33. (canceled)

34. The method of claim 1, wherein the characterizing the conduit is implemented using a classification-based machine learning algorithm using training data labeled with characterizations of the conduit.

35-37. (canceled)

38. The method of claim 8, wherein the flow rate value of the first airflow and the second airflow is generated by a flow sensor communicatively coupled to the respiratory therapy device and wherein the pressure value of the first airflow and the second airflow is generated by a pressure sensor communicatively coupled to the respiratory therapy device.

39-40. (canceled)

41. The method of claim 1, wherein the first airflow is a laminar flow of air having a Reynolds number of less than about 2300.

42. The method of claim 1, wherein the first airflow has an associated noise level of no more than about 40 dBA measured at a distance of about one meter from the respiratory therapy device.

43. The method of claim 1, wherein the first airflow has a flow rate value of between about 0.1 and 0.8 liters per second.

44. The method of claim 1, wherein the motor of the respiratory therapy device operates between about 3,000 revolutions per minute and about 12,000 revolutions per minute.

45. The method of claim 44, further comprising:
determining, based on characterizing the conduit, a third airflow parameter value for prospective delivery of pressurized airflow to a user, when the user wears the user interface for respiratory therapy, wherein the determining the third airflow parameter value includes determining impedance due to (i) intentional leak through one or more vents in the user interface, (ii) unintentional leak at the user interface or a mouth of the user, or (iii) a combination of both.

46. (canceled)

47. The method of claim 44, wherein:
the user interface includes an anti-asphyxia valve; and
the first airflow has a flow rate value of less than about 0.4 liters per second.

48. The method of claim 1, wherein the respiratory therapy device includes a waterless humidifier, the method further comprising determining a type of the waterless humidifier based on the first relationship.

49-52. (canceled)

53. A system comprising:
a respiratory therapy device configured to supply pressurized air during a sleep session of a user;
one or more sensors configured to detect airflow parameter data associated with the supplied pressurized air; and
a control system including one or more processors configured to execute machine-readable instructions to:
generate a first airflow from the respiratory therapy device to the conduit by operating a motor of the respiratory therapy device at a first revolutions per minute;
receive a first airflow parameter data associated with the first airflow, the first airflow parameter data comprising a first pair of two distinct airflow parameter values;
determine a first relationship between the first airflow parameter data and the conduit; and
characterize the conduit based on the first relationship.

54-100. (canceled)

101. A non-transitory machine readable medium having stored thereon instructions for performing a method comprising machine executable code which when executed by at least one machine, causes the machine to:
generate a first airflow from the respiratory therapy device to the conduit by operating a motor of the respiratory therapy device at a first revolutions per minute;
receive a first airflow parameter data associated with the first airflow, the first airflow parameter data comprising a first pair of two distinct airflow parameter values;
determine a first relationship between the first airflow parameter data and the conduit; and
characterize the conduit based on the first relationship.
